(19) United States
(12) Patent Application Publication
(10) Pub. No.: US 2013/0138006 A1
(43) Pub. Date: May 30, 2013

(54) SINGLE CHAMBER LEADLESS INTRA-CARDIAC MEDICAL DEVICE HAVING DUAL CHAMBER SENSING WITH SIGNAL DISCRIMINATION

(71) Applicants: Gene A. Bornzin, Simi Valley, CA (US); Xiaoyi Min, Thousand Oaks, CA (US); John W. Poore, South Pasadena, CA (US); Zoltan Somogyi, Simi Valley, CA (US)

(72) Inventors: Gene A. Bornzin, Simi Valley, CA (US); Xiaoyi Min, Thousand Oaks, CA (US); John W. Poore, South Pasadena, CA (US); Zoltan Somogyi, Simi Valley, CA (US)

(73) Assignee: PACESETTER, INC., Sylmar, CA (US)

(21) Appl. No.: 13/485,534

(22) Filed: May 31, 2012

Related U.S. Application Data
(60) Provisional application No. 61/555,973, filed on Nov. 4, 2011.

(51) Int. Cl. A61B 5/042

(52) U.S. Cl. CPC A61B 5/042
USPC 600/509

(57) ABSTRACT

A leadless intra-cardiac medical device (LIMD) includes multiple electrodes that allow for stimulation and sensing of the right ventricle (RV) and sensing of the right atrium (RA), even though it is entirely located in the RV. The LIMD includes a housing having a proximal end configured to engage local tissue in the local chamber and electrodes located at multiple locations along the housing. Sensing circuitry is configured to define a far field (FF) channel between a first combination of the electrodes to sense FF signals occurring in the adjacent chamber. The sensing circuitry is configured to define a near field (NF) channel between a second combination of the electrodes to sense NF signals occurring in the local chamber. A controller is configured to analyze the NF and FF signals to determine whether the NF and FF signals collectively indicate that a validated event of interest occurred in the adjacent chamber.

### CROSS REFERENCE TO RELATED APPLICATIONS

This application relates to and claims priority benefits from U.S. Provisional Application No. 61/555,973, filed Nov. 4, 2011, entitled “Single Chamber Leadless Implantable Medical Device Having Dual Chamber Sensing With Signal Discrimination,” which is hereby incorporated by reference in its entirety. This application also relates to U.S. patent application Ser. No. 13/352,048, filed Jan. 17, 2012, entitled “Single-Chamber Leadless Intra-Cardiac Medical Device with Dual-Chamber Functionality”; and Ser. No. 13/352,136, filed Jan. 17, 2012, entitled “Dual-Chamber Leadless Intra-Cardiac Medical Device with Intra-Cardiac Extension”; and ______, filed ______, entitled “Leadless Intra-Cardiac Medical Device With Dual Chamber Sensing Through Electrical and/or Mechanical Sensing” (Atty Docket No. A12P1029), which are hereby incorporated by reference in their entirety.

### FIELD OF THE INVENTION

Embodiments of the present invention generally relate to leadless implantable medical devices, and more particularly to leadless intra-cardiac medical devices that afford dual chamber sensing from a position within a single chamber of the heart and with signal discrimination from a position within a single chamber of the heart. As used herein, the term “leadless” generally refers to an absence of electrically-conductive leads that traverse vessels or other anatomy outside of the intra-cardiac space, while “intra-cardiac” means generally, entirely within the heart and associated vessels, such as the SVC, IVC, CS, pulmonary arteries and the like.

### BACKGROUND OF THE INVENTION

Current implantable medical devices (IMD) for cardiac applications, such as pacemakers, include a “housing” or “can” and one or more electrically-conductive leads that connect to the can through an electro-mechanical connection. The can is implanted outside of the heart, in the pectoral region of a patient and contains electronics (e.g., a power source, microprocessor, capacitors, etc.) that provide pacemaker functionality. The leads traverse blood vessels between the can and heart chambers in order to position one or more electrodes carried by the leads within the heart, thereby allowing the device electronics to electrically excite or pace cardiac tissue and measure or sense myocardial electrical activity.

To sense atrial cardiac signals and to provide right atrial chamber stimulation therapy, the can is coupled to an implantable right atrial lead including at least one atrial tip electrode that typically is implanted in the patient's right atrial appendage. The right atrial lead may also include an atrial ring electrode to allow bipolar stimulation or sensing in combination with the atrial tip electrode.

Before implantation of the can into a subcutaneous pocket of the patient, however, an external pacing and measuring device known as a pacing system analyzer (PSA) is used to ensure adequate lead placement, maintain basic cardiac functions, and evaluate pacing parameters for an initial programming of the IMD. In other words, a PSA is a system analyzer that is used to test an implantable device, such as an implantable pacemaker.

To sense the left atrial and left ventricular cardiac signals and to provide left-chamber stimulation therapy, the can is coupled to the “coronary sinus” lead designed for placement in the “coronary sinus region” via the coronary sinus ostium in order to place a distal electrode adjacent to the left ventricle and additional electrode(s) adjacent to the left atrium. As used herein, the phrase “coronary sinus region” refers to the venous vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus.

Accordingly, the coronary sinus lead is designed to: receive atrial and/or ventricular cardiac signals; deliver left ventricular pacing therapy using at least one left ventricular tip electrode for unipolar configurations or in combination with left ventricular ring electrode for bipolar configurations; deliver left atrial pacing therapy using at least one left atrial ring electrode as well as shocking therapy using at least one left atrial coil electrode.

To sense right atrial and right ventricular cardiac signals and to provide right-chamber stimulation therapy, the can is coupled to an implantable right ventricular lead including a right ventricular (RV) tip electrode, a right ventricular ring electrode, a right ventricular coil electrode, a superior vena cava (SVC) coil electrode, and so on. Typically, the right ventricular lead is inserted transvenously into the heart so as to place the right ventricular tip electrode in the right ventricular apex such that the RV coil electrode is positioned in the right ventricle and the SVC coil electrode will be positioned in the right atrium and/or superior vena cava. Accordingly, the right ventricular lead is capable of receiving cardiac signals, and delivering stimulation in the form of pacing and shock therapy to the right ventricle.

Although a portion of the leads, as well as the IMD itself are outside of the patient's heart. Consequently, bacteria and the like may be introduced into the patient's heart through the leads, as well as the IMD, thereby increasing the risk of infection within the heart. Additionally, because the IMD is outside of the heart, the patient may be susceptible to Twiddler's syndrome, which is a condition caused by the shape and weight of the IMD itself. Twiddler's syndrome is typically characterized by a subconscious, inadvertent, or deliberate rotation of the IMD within the subcutaneous pocket formed in the patient. In one example, a lead may retract and begin to wrap around the IMD. Also, one of the leads may dislodge from the endocardium and cause the IMD to malfunction. Further, in another typical symptom of Twiddler's syndrome, the IMD may stimulate the diaphragm, vagus, or phrenic nerve, pectoral muscles, or brachial plexus. Overall, Twiddler's syndrome may result in sudden cardiac arrest due to conduction disturbances related to the IMD.

In addition to the foregoing complications, leads may experience certain further complications, such as incidences of venous stenosis or thrombosis, device-related endocarditis, lead perforation of the tricuspid valve and concomitant tricuspid stenosis; and lacerations of the right atrium, superior vena cava, and innominate vein or pulmonary embolization of electrode fragments during lead extraction.

To combat the foregoing limitations and complications, small sized devices configured for intra-cardiac implant have been proposed. These devices, termed leadless pacemakers (LLPM) are typically characterized by the following features: they are devoid of leads that pass out of the heart to another component, such as a pacemaker outside of the heart; they include electrodes that are affixed directly to the “can” of the device; the entire device is attached to the heart; and the device is capable of pacing and sensing in the chamber of the heart where it is implanted.

LLPM devices that have been proposed thus far offer limited functional capability. These LLPM devices are able to sense in one chamber and deliver pacing pulses in that same chamber, and thus offer single chamber functionality. For example, an LLPM device that is located in the right atrium would be limited to offering AAI mode functionality. An AAI mode LLPM can only sense in the right atrium, pace in the right atrium and inhibit pacing function when an intrinsic event is detected in the right atrium within a preset time limit. Similarly, an LLPM device that is located in the right ventricle would be limited to offering VVI mode functionality. A WI mode LLPM can only sense in the right ventricle, pace in the right ventricle and inhibit pacing function when an intrinsic event is detected in the right ventricle within a preset time limit. To gain widespread acceptance by clinicians, it would be highly desired for LLPM devices to have dual chamber pacing/sensing capability (DDD mode) along with other features, such as rate adaptive pacing.

It has been proposed to implant sets of multiple LLPM devices within a single patient, such as one or more LLPM devices located in the right atrium and one or more LLPM devices located in the right ventricle. The atrial LLPM devices and the ventricular LLPM devices wirelessly communicate with one another to convey pacing and sensing information there between to coordinate pacing and sensing operations between the various LLPM devices.

However, these sets of multiple LLPM devices experience various limitations. For example, each of the LLPM devices must expend significant power to maintain the wireless communications links. The wireless communications links should be maintained continuously in order to constantly convey pacing and sensing information between, for example, atrial LLPM device(s) and ventricular LLPM device(s). This pacing and sensing information is necessary to maintain continuous synchronous operation, which in turn draws a large amount of battery power.

Further, it is difficult to maintain a reliable wireless communications link between LLPM devices. The LLPM devices utilize low power transceivers that are located in a constantly changing environment within the associated heart chamber. The transmission characteristics of the environment surrounding the LLPM device change due in part to the continuous cyclical motion of the heart and change in blood volume. Hence, the potential exists that the communications link is broken or intermittent.

A need remains for an improved pacer for location in a single chamber, such as the RV, that provides ventricular pacing/sensing, and atrial sensing capabilities. The need remains for a simplified system that retains a single leadless pacer inside RV with the feature of atrial sensing that would be applicable to patients with heart block.

### SUMMARY OF THE INVENTION

A leadless intra-cardiac medical device (LIMD) is provided with dual chamber sensing, without leads, despite the fact that the entire device is located in one chamber. In one embodiment, the LIMD includes multiple electrodes that allow for stimulation and sensing of the right ventricle (RV) and sensing of the right atrium (RA), even though it is entirely located in the RV. The electrodes include a dual purpose intermediate electrode positioned between a distal electrode and a proximal electrode. The LIMD is sized such that when the proximal electrode is positioned in the region of the RV apex, the distal electrode is in the region of an atrial or ventricular valve. In this arrangement, the intermediate electrode and proximal electrode provide ventricular pacing and sensing, while the intermediate electrode in combination with the distal atrial electrode provides for atrial sensing. The LIMD also includes an algorithm process that discriminates between valid and invalid atrial events and ventricular events.

In accordance with an embodiment, an LIMD is provided that is configured to be implanted entirely within a single local chamber of the heart and remote from an adjacent chamber of the heart. The LIMD includes a housing having a proximal end configured to engage local tissue of interest in the local chamber, a distal end, and electrodes located at multiple locations along the housing. Sensing circuitry is configured to define a far field (FF) channel between a first combination of the electrodes to sense FF signals occurring in the adjacent chamber. The sensing circuitry is configured to define a near field (NF) channel between a second combination of the electrodes to sense NF signals occurring in the local chamber. A controller configured to analyze the NF and FF signals to determine whether the NF and FF signals collectively indicate that a validated event of interest occurred in the adjacent chamber.

The electrodes include a proximal electrode located at the proximal end, a distal electrode located at the distal end and an intermediate electrode located at an intermediate region along the housing. In one arrangement, the first electrode combination includes the distal electrode and the intermediate electrode. In another arrangement, the second electrode combination comprises the proximal electrode and the intermediate electrode. The proximal and intermediate electrodes are separated by a first inter-electrode (IE) spacing, the distal and intermediate electrodes are separated by a second IE spacing, and the second IE spacing is greater than the first IE spacing.

The controller may be configured to compare the FF signals sensed over the FF channel to a FF adjacent-chamber criteria, compare the NF signals sensed over the NF channel to a NF adjacent-chamber criteria, and declare a validated event when both of the criteria are satisfied. The FF adjacent-chamber criteria may include a FF signal amplitude threshold and the NF adjacent-chamber criteria may be a NF signal amplitude threshold, in which case the criteria may be satisfied when the amplitude of the sensed FF signals exceeds the FF signal amplitude threshold and the amplitude of the sensed NF signals does not exceed the NF signal amplitude threshold.

Alternatively, or additionally, the FF adjacent-chamber criteria may include a FF signal morphology and the NF adjacent-chamber criteria may include a NF signal morphology, in which case the criteria may be satisfied when the morphology of the sensed FF signals matches the FF signal morphology and the morphology of the sensed NF signals matches the NF signal morphology. Alternatively, or additionally, the FF adjacent-chamber criteria may include a FF signal amplitude range and the NF adjacent-chamber criteria may include a NF signal amplitude threshold, in which case the criteria is satisfied when the amplitude of the sensed FF signals is within the FF signal amplitude range and the amplitude of the sensed NF signals does not exceed the NF signal amplitude threshold.

The controller may be further configured to analyze the NF and FF signals to determine whether the NF and FF signals collectively indicate that a validated event of interest occurred in the local chamber. In this embodiment, the controller may be configured to compare the FF signals sensed over the FF channel to a FF local-chamber criteria, compare the NF signals sensed over the NF channel to a NF local-chamber criteria, and declare a validated event when both of the criteria are satisfied. In one configuration, the FF local-chamber criteria may be a FF signal amplitude threshold and the NF local-chamber criteria may be a NF signal amplitude threshold, in which case the criteria is satisfied when the amplitude of the sensed FF signals exceeds the FF signal amplitude threshold and the amplitude of the sensed NF signals exceeds the NF signal amplitude threshold.

Alternatively, or additionally, the FF local-chamber criteria may be a FF signal morphology, the NF local chamber criteria may be a NF signal morphology, in which case the criteria is satisfied when the morphology of the sensed FF signals matches the FF signal morphology and the morphology of the sensed NF signals match the NF signal morphology.

In accordance with an embodiment, a method is provided to sense cardiac activity from an LIMD configured to be implanted entirely within a single local chamber of the heart and remote from an adjacent chamber. The method comprised of sensing far field (FF) signals over a FF channel between a first combination of electrodes provided on the LIMD in the local chamber and sensing near field (NF) signals over a NF channel between a second combination of electrodes provided on the LIMD in the local chamber. The method analyzes both of the NF and FF signals to determine whether the NF and FF indicate that event of interest occurred in the adjacent chamber.

### BRIEF DESCRIPTION OF THE DRAWINGS

FIG. 1 illustrates a sectional view of the patient's heart and shows a leadless intra-cardiac medical device.

FIG. 2 illustrates a processing sequence carried out to establish thresholds and/or morphology templates.

FIG. 3A illustrates a general process flow for analyzing near-field (NF) and far-field (FF signals.

FIG. 3B illustrates exemplary local-chamber-sensed NF and FF signals with respect to adjacent-chamber criteria, in the form of amplitude thresholds, for use in validating adjacent-chamber events.

FIG. 4A illustrates a processflow for validating local-chamber events using NF and FF signals sensed in the local chamber.

FIG. 4B illustrates exemplary local-chamber NF and FF signals with respect to local-chamber criteria, in the form of amplitude thresholds, for use in validating local-chamber events.

FIG. 5A illustrates an exemplary process flow for validating adjacent-chamber events using NF and FF signals sensed in the local chamber.

FIG. 5B illustrates exemplary local-chamber-sensed NF and FF signals with respect to adjacent-chamber criteria, in the form of amplitude thresholds, for use in validating adjacent-chamber events.

FIG. 6 illustrates an exemplary timing diagram for various sensed signals, sensing windows, RP delays and PR delays.

FIG. 7 illustrates an leadless intra-cardiac medical device (LIMD).

FIG. 8 shows an exemplary LIMD 800 configured for dual-chamber functionality from a primary location within a single chamber of the heart.

### DETAILED DESCRIPTION

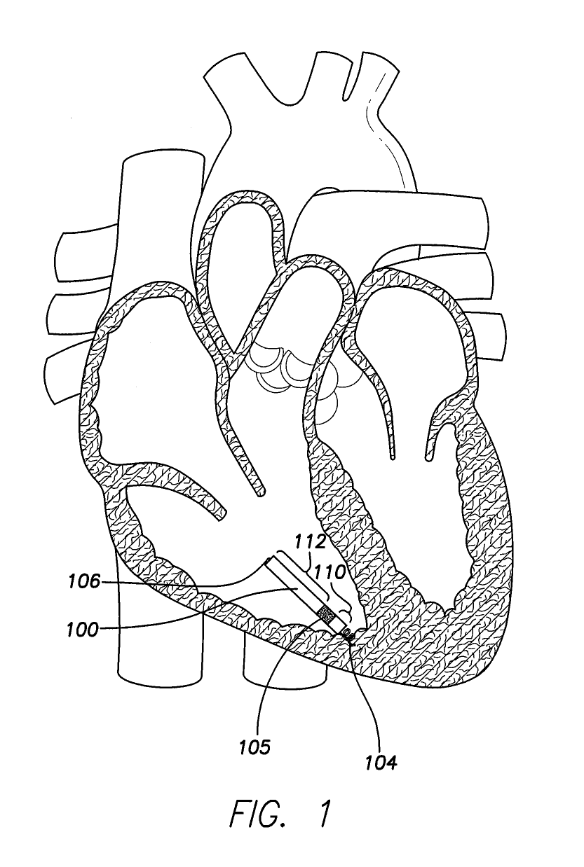

FIG. 1 provides a sectional view of the patient's heart and shows a leadless intra-cardiac medical device (LIMD) 100 implanted in the area of the right ventricular apex. In this arrangement, the LIMD 100 is a VDD pacer located entirely inside the right ventricle (RV). The LIMD 100 provides for detection of ventricular electrical cardiac events through near-field bipolar sensing in the area of the RV apex, and for detection of atrial electrical cardiac events through enhanced atrial far-field sensing in a region generally near an atrial or ventricular valve, such as the area below the tricuspid valve. The enhanced sensing is provided by an arrangement of electrodes that include a proximal electrode 104, an intermediate electrode 105, and a distal electrode 106. An inter-electrode (IE) spacing 110 between the proximal electrode 104 and the intermediate electrode 105 is configured to allow for far field detection of atrial events, while the inter-electrode spacing 112 between the distal electrode 106 and the intermediate electrode 105 is configured to allow for sensing of near-field ventricular events and rejection of far-field atrial signals.

In one embodiment, the proximal electrode 104 is provided in the form of a helix, the intermediate electrode 105 is provided in the form of a ring and the distal electrode 106 is provided in the form of a dome, bump or button. Other electrode configurations are possible. For example, while the proximal electrode 104 is a helix that provides for active fixation into myocardium, the helix configuration may be replaced with a passive electrode configuration, e.g., a dome tip electrode with fixation tines, a straight needle or pin, a bump electrode, a rounded tip electrode and the like.

As an example, the IE spacing 110 between a leading edge of the intermediate electrode 105 and a trailing edge of the proximal electrode 104 may be less than 5 mm, and approximately between 1-2 mm. The IE spacing 110 is set to a distance sufficient to afford sensing, between the intermediate and proximal electrodes 105, 104 of near-field ventricular events with rejection of far-field atrial signals. The intermediate and proximal electrodes 105, 104 may be configured to perform bipolar sensing of near-field ventricular events with rejection of far-field atrial signals.

The IE spacing 112 between a leading edge of the distal electrode 106 and a trailing edge of the intermediate electrode 105 may be at least 20 mm, and may be approximately 20-40, or 30 mm or greater. The IE spacing 112 is set to a distance sufficient to afford far-field detection of atrial events between the distal electrode 106 and the intermediate electrode 105. The distal electrode 106 and the intermediate electrode 105 may be configured to perform bipolar sensing of near-field ventricular events with rejection of far-field atrial signals. In operation, the intermediate electrode 105 may be configured as an anode, with each of the respective distal electrode 106 and proximal electrode 104 configured as cathodes. The proximal and intermediate electrodes 104, 105 form an atrial sensing channel (also referred to as a far-field sensing channel or far-field sensing electrode pair), while the distal and intermediate electrodes 106, 105 form a ventricular sensing channel (also referred to as a near-field sensing channel or near-field sensing electrode pair).

In order to avoid detection of ventricular events, the atrial sensing channel is activated only for a period of time during each cardiac cycle. This period of time is referred to as an atrial sensing window. Even so, given the far-field arrangement of the atrial sensing channel, it is possible for either of true (or valid) atrial events, e.g., signals originating from an atrial depolarization (P-wave), or false (or invalid) atrial events, e.g., signals originating from somewhere other than an atrial depolarization, such as a premature ventricular contraction (PVC), to be detected during the atrial sensing window.

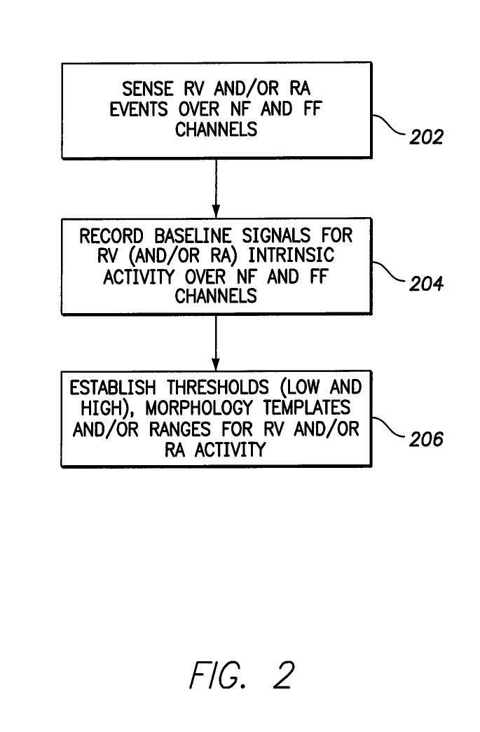

FIG. 2 illustrates a computer implemented processing sequence carried out in accordance with an embodiment to establish thresholds, ranges and/or morphology templates. Beginning at 202, the near field (NF) and far field (FF) sensing circuitry collect baseline NF and FF signals over the NF and FF channels, respectively. The NF and FF baseline signals are collected over at least a portion of one or more cardiac cycles during which intrinsic physiologic (e.g. normal, healthy) baseline activity of interest occurs. The activity of interest may occur in the right atrium (RA), right ventricle (RV), left atrium (LA) or left ventricle (LV), or any combination thereof. For example, the NF and FF baseline signals may be collected over the portion of a single or over multiple cardiac cycles that corresponds to the QRS complex, thereby collecting NF and FF baseline signals representative of intrinsic physiologic R-waves. As a further example, the NF and FF baseline signals may be collected over the portion of a single or over multiple cardiac cycles that corresponds to the ST segment. As a further example, the NF and FF baseline signals may be representative of intrinsic physiologic P-waves. Optionally, the NF and FF baseline signals may be collected over one or more complete cardiac cycles during which intrinsic physiologic (e.g. normal, healthy) baseline activity occurs. The NF and FF baseline signals may be representative of intrinsic physiologic R-waves and P-waves.

At 204, the baseline signals are recorded for the intrinsic activity of interest. For example, the baseline signals may be recorded for intrinsic physiologic activity in the RV, and/or intrinsic physiologic activity in the RA.

At 206, the recorded NF and FF baseline signals are analyzed to establish automatically one or more thresholds, ranges, morphology models or templates, and the like. The NF and FF baseline signals are used to discriminate between valid and invalid “remote” events of interest that originate in an adjacent chamber, such as during detection of physiologic intrinsic atrial events (P-waves) over the FF (atrial) channel. The LIMD 100 automatically establishes one or more thresholds related to sensed “local” (e.g. ventricular) events. For example, the baseline recordings (over the NF channel) may represent ventricular sensed events, from which peaks of the QRS complex are obtained. The peaks of the QRS complex are then used to establish a low threshold (T_{LOW}) and a high threshold (T_{HIGH}). For example, a low threshold may be automatically set as a percentage of the peak values of the QRS complex sensed over the NF channel. The low threshold (T_{LOW}), also referred to as the remote signal NF threshold, represents a limit associated with the NF channel (e.g., the ventricular channel) that is used in connection with validating far field events (e.g., atrial P-waves). As explained herein, when a NF signal, that is collected over the NF channel, has an amplitude that exceeds the T_{LOW} (the NF threshold), this is an indicator that the NF signal is too large to be associated with a valid “remote” event (P-wave). As one example, the low threshold (T_{LOW}) may be automatically set at 10% or 20% of the peak of the QRS complex as measured over the NF channel (ventricular channel).

The high threshold (T_{HIGH}), also referred to as the remote signal FF threshold, represents a limit associated with the FF channel (e.g., the atrial channel) that is used in connection with validating far field events (e.g., atrial P-waves). As explained herein, when a FF signal, that is collected over the FF channel, has an amplitude that falls below the T_{HIGH} (FF threshold), this is an indicator that the FF signal is too small to be associated with a valid remote event (P-wave). As one example, the high threshold (T_{HIGH}) may be set at 80% of the QRS complex as measured over the FF channel.

Optionally, the process of FIG. 2 may be modified to calculate automatically acceptable amplitude ranges or morphology templates for the NF signal and FF signals. For example, the range for the FF signal may be 10-25% of the QRS peak and the range for the NF signal may be 75-90% of the QRS peak.

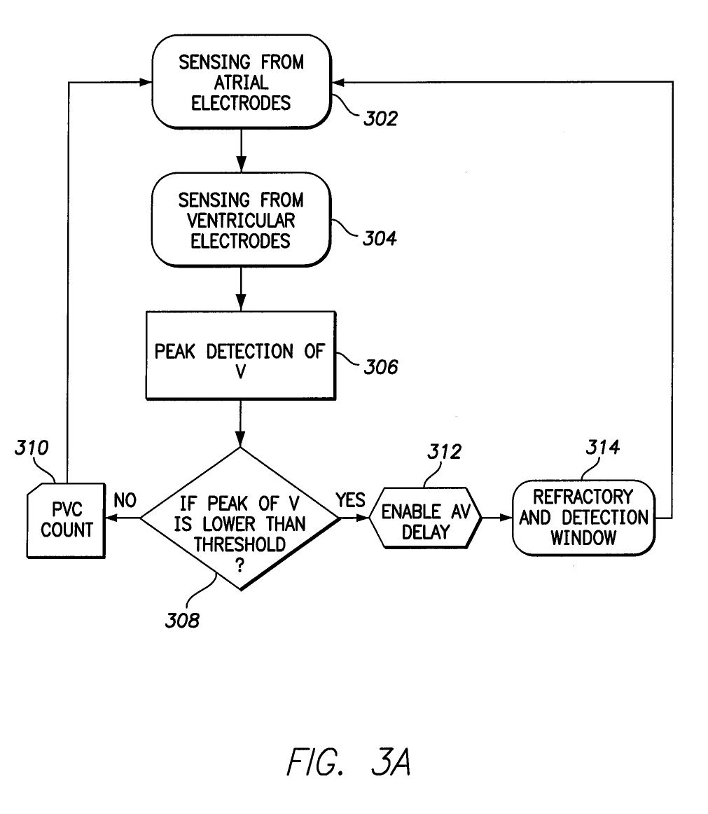

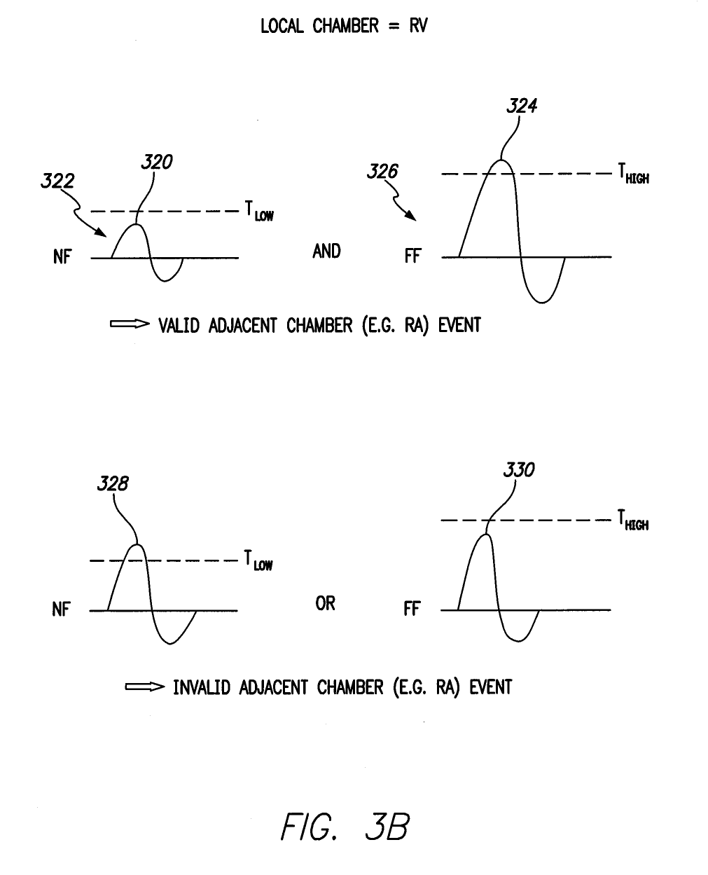

FIG. 3A illustrates a computer implemented general process flow in accordance with an embodiment. As explained hereafter, embodiments are provided in which, once thresholds are established, NF and FF signals are sensed (at 302 and 304) using the atrial or FF sensing channel and the ventricular or NF sensing channel. At 306, the NF signal over the NF channel (also referred to in this example as a ventricular channel) is analyzed to detect the peak of the NF signal. The NF signals sensed by the NF channel, i.e., sensed V signals, are used to discriminate between valid and invalid atrial events sensed by the atrial or FF channel, i.e., sensed A signals.

At 308, and with additional reference to FIG. 3B, it is determined whether the peak 320 of the NF signal 322 over the NF channel (e.g., ventricular channel) is lower than the remote signal NF threshold (T_{LOW}) and if the peak 324 of the FF signal 326 sensed over the FF channel (e.g., atrial channel) is greater than the high threshold (T_{HIGH}). If both the peak 320 of NF signal is less than the low threshold (T_{LOW}) and the peak 324 of the FF signal is greater than the high threshold (T_{HIGH}), the signal 326 sensed over the FF channel (e.g., atrial channel) is considered a valid atrial signal, (e.g., P-wave). However, if either the peak 328 of NF signal is greater than the low threshold (T_{LOW}), or the peak 330 of the FF signal sensed over the FF channel (e.g., atrial channel) is less than the high threshold (T_{HIGH}), the FF signal is considered an invalid atrial event, such as a premature ventricular contraction (PVC).

If the test at 308 is positive (YES) to valid a P-wave, then an atrio-ventricular (AV) delay is enabled at 312. At the end of the AV delay, if no intrinsic ventricular event occurs, then a ventricular pacing stimulus is delivered such as from the intermediate-proximal electrode pair. After the ventricular pacing stimulus is delivered, at 314 capture is confirmed and a refractory period starts in both the FF channel (atrial channel) and the NF channel (ventricular channel), and the process returns to 302 to start a search window to sense at both the FF channel and the NF channel. During the refractory period (referred to as the post ventricular atrial refractory period or PVARP), the sensing circuitry is blocked to disable sensing. It should also be understood that, when a normal intrinsic ventricular event is detected, the sensing circuitry is blocked for the PVARP interval.

Returning to 306, if an invalid atrial event was declared, then flow moves to 310 where a PVC counter is incremented and the process returns to 302 to start a search window to sense at both the FF channel and the NF channel. Optionally, at 310, a pacing therapy may be delivered, such as in response to detecting a select number of PVCs.

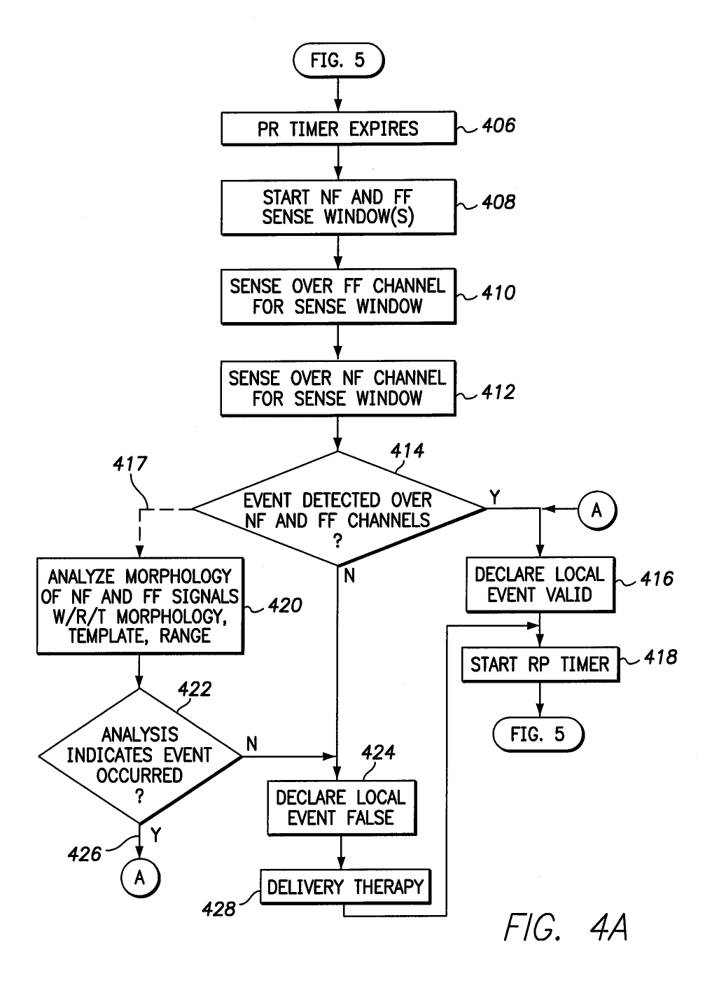

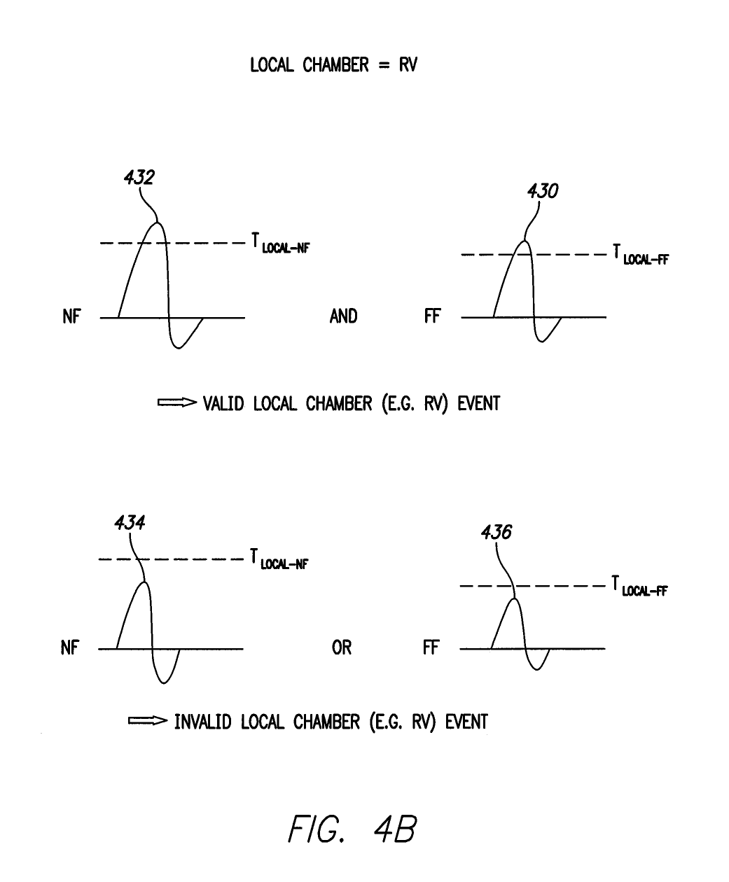

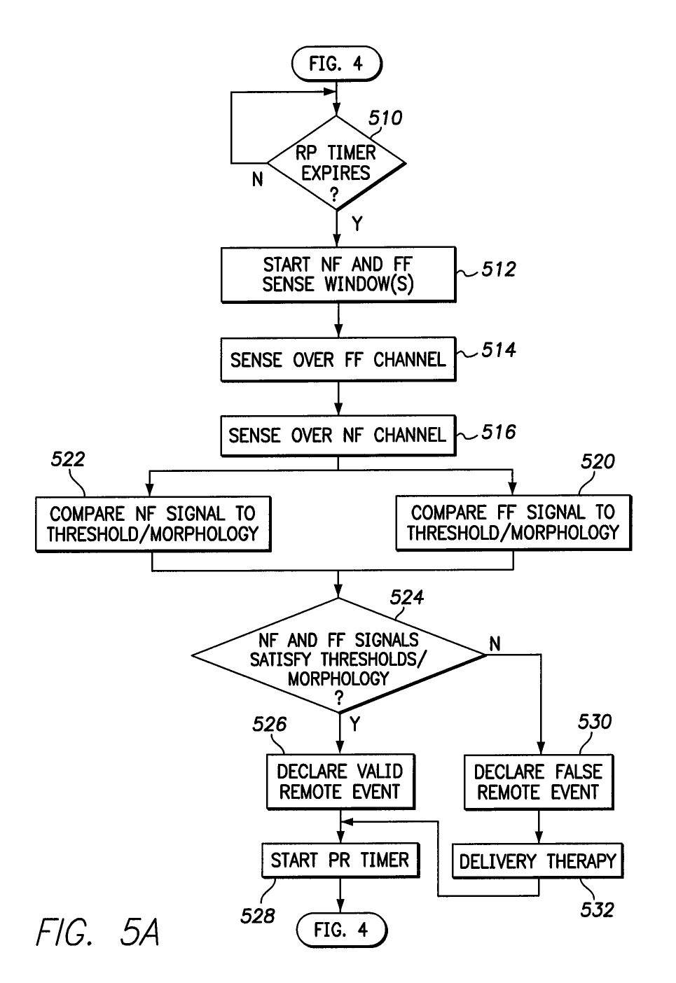

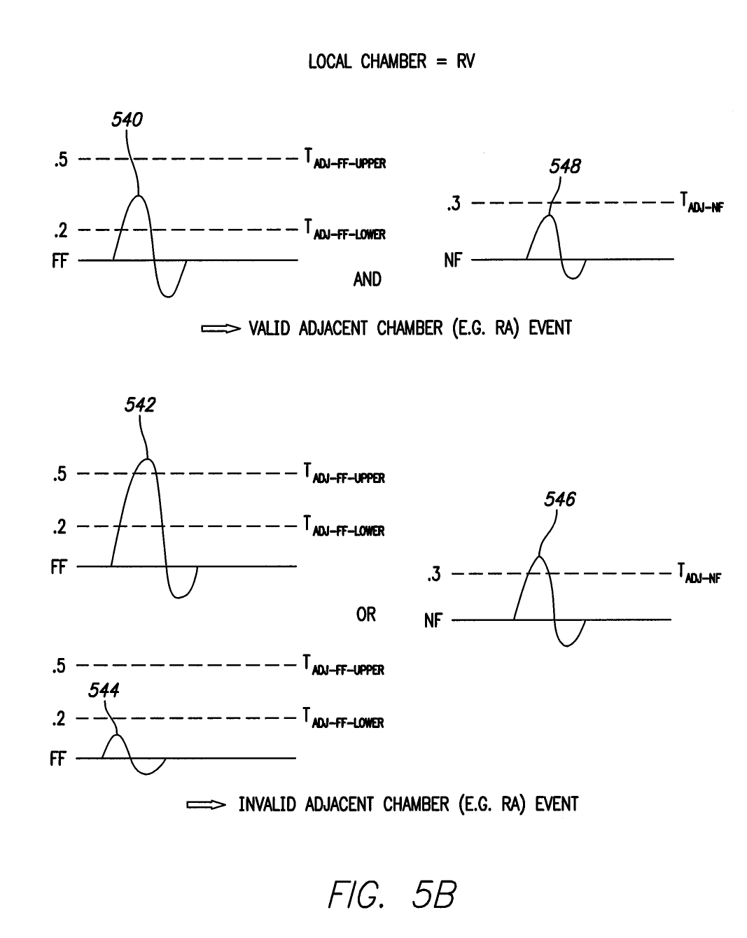

FIGS. 4A and 5A illustrate a computer implemented processing sequence carried out in accordance with an embodiment.

The process of FIGS. 4A and 5A is discussed for an embodiment that uses a pair of sensing channels defined by electrodes in a local chamber. The process of FIG. 4A is used to determine whether a valid event of interest occurs in the “local” chamber (e.g. ventricle) where the electrodes are located. The process of FIG. 5A is used to determine whether a valid event of interest occurs in the “adjacent” chamber (e.g. atrium) remote from where the electrodes are not located. The following discussion of FIGS. 4A and 5A will include examples for an LIMD 100 that is located within the right ventricle as the local chamber. However, it is understood that the LIMD 100 may be implanted within any chamber of the heart which then would constitute the “local” chamber, while the other three chambers of the heart would constitute “adjacent” chambers. Hence, if the LIMD 100 is implanted in the left ventricle, then the LV is the local chamber, while the RV, RA and LA would represent adjacent chambers. Similarly, if the LIMD 100 is implanted in the left atrium, then the LA is the local chamber, while the RV, RA and LV represent adjacent chambers.

In general, the process of FIG. 4A begins at the point in a cardiac cycle after an intrinsic or paced atrial P-wave occurs and the PR timer expires. The process of FIG. 4A seeks to validate a ventricular event (which occurs in the local chamber where the LIMD is located) based on signals sensed over both sensing channels, i.e., the NF channel and FF channel. Beginning at 406, a timer expires such as a PR timer expiring. At 408, NF and FF sensing windows are activated for the FF and NF channels. The NF and FF sensing windows may be activated for both of the FF and NF sensing circuitry simultaneously by the controller in order to begin to collect NF and FF signals at the same time and end at the same or different times. A single common sensing window may be used for the NF and FF channels. Optionally, the NF and FF sensing windows may be activated at different points in time, where the NF and FF sensing window activation times are slightly offset or staggered such that the NF and FF sensing windows only partially overlap.

The sensing windows may be activated at a predetermined point in time following a select cycle reset event. For example, the select cycle reset event may represent a ventricular contraction (R-wave), a ventricular paced event, a programmed R-R interval timer and the like.

While the sensed signals are referred to as NF and FF signals, it is understood that the actual origin of a cardiac event that causes a NF signal and FF signal may or may not originate in the remote far field (i.e. adjacent chamber) or may or may not originate in the local near field (i.e. local chamber). Instead, the “NF” and “FF” designators indicate that the corresponding channel is tuned to be sensitive to, or “listen for”, signals that are expected to originate at a near or local site (for the NF channel) or to originate at a far or remote site (for the FF channel).

At 410, the FF sensing circuitry in the LIMD 100 senses electrical signals over a channel tuned to listen for activity originating in the far field (FF) for the duration of the FF sensing window. Optionally, if the LIMD 100 is implanted in the left ventricle, then the FF channel may be tuned to listen for activity originating in the RV, RA and LA which represent adjacent chambers. Similarly, if the LIMD 100 is implanted in the left atrium, then the FF channel may be tuned to listen to the RV, RA and LV which represent adjacent chambers.

At this point in the cardiac cycle (after the PR timer expires), the FF signals sensed over the FF channel are not expected to be representative of activity originating in an adjacent chamber. Instead the FF signals are expected to be representative of cardiac events or cardiac activity that occurs intrinsically or that corresponds to a paced event in the local chamber. The LIMD 100 listens over the FF channel even though a P-wave is not expected. At this stage in the cardiac cycle, the FF channel (while still tuned to listen in the far field) is expected to sense activity originating in the local chamber (i.e., an R-wave), and produce a FF signal that is commensurate in shape and/or size to a healthy normal R-wave.

At 412, the NF sensing circuitry in the LIMD 100 senses electrical signals over a NF channel tuned to listen for activity in a ventricle, for the duration of the NF sensing window. The NF and FF sensing windows may have the same duration. Optionally, the NF and FF sensing windows may differ in length such that one or the other of the NF and FF channels collect sensed signals for a period of time longer than the other of the NF and FF channels. The sensing operations as 410 and 412 are performed simultaneously, or during at least partially overlapping sensing windows, such that the signals sensed over the FF channel and over the NF channel correspond to (and originate from) a same or common cardiac event. The common cardiac event is expected to originate in the local chamber, but during an arrhythmia may originate in an adjacent chamber. As explained hereafter, the FF and NF signals are both analyzed to determine the origin.

The FF signals are sensed by a FF channel that includes a first electrode combination that is provided on or near the LIMD 100 in the local chamber. The NF signals are sensed by a NF channel that includes a second electrode combination that is provided on or near the LIMD 100 in the local chamber. The first and second electrical combinations may at least partially overlap (e.g., use a common electrode). For example, the first electrode combination (FF channel) may include an electrode pair such as the distal electrode 106 and the intermediate electrode 105. The second electrode combination (NF channel) may include another electrode pair such as the proximal electrode 104 and the intermediate electrode 105. Optionally, one or both of the first and second electrode combinations may include other single electrodes, pairs of electrodes, or sets of more than two electrodes. For example, the LIMD 100 may be provided with electrodes located in different locations on, proximate to, or remote from, the housing. The electrode combinations may include supplemental or substitute electrodes in addition to the electrodes 104-106 shown in FIG. 1. When additional electrodes are provided beyond electrodes 104-106, these additional electrodes may be used in place of, or in combination with, the electrodes 104-106. The electrode combinations used to provide sensing may be the same as, or different from, the electrode combinations used to deliver stimulus for therapy.

NF and FF sensing circuitry collect NF and FF signals over the NF and FF sensing window(s), respectively. Once the FF and NF sensing windows expire, the FF and NF sensing circuitry is de-activated by the controller. The duration of the FF sensing windows may be the same as, or differ from, the duration of the NF sensing window. The NF and FF signals may be stored in the device memory or conveyed to a buffer or short-term memory in the controller.

At 414, the controller analyzes the FF and NF signals to determine whether the FF and NF signals indicate that a validated event of interest (in the local chamber) was detected over one or both of the FF (e.g., atrial) and NF (e.g., ventricular) channels. The determination at 414 may be based on comparison of the peaks or shapes of the NF and FF signals to thresholds, ranges, morphologies and the like.

The FF signals, when valid, are representative of cardiac activity occurring in the local chamber. For example, with reference to FIG. 4B, an intrinsic normal ventricular event is sensed over the FF channel 430 as a large R-wave and has a shape and/or amplitude that satisfies a criteria, e.g., is within an acceptable range, above a certain threshold (T_{LOCAL-FF}), or with an acceptable morphology. The same intrinsic normal ventricular event is sensed over the NF channel 432 as a NF signal and has a shape and/or amplitude within an acceptable range, above a certain threshold (T_{LOCAL-NF}) or with an acceptable morphology. For example, the ventricular event may cause a FF signal (as sensed from a FF channel by electrodes in the ventricle) that has a peak of X millivolts and a NF signal (as sensed from a NF channel by electrodes in a ventricle) that has a peak of Y millivolts. The FF signal, when from a ventricular event, is large and the NF signal, even when from a ventricular event, is also relatively large. At 414, the controller compares the NF and FF signals to acceptable (e.g., present programmed, automatically set) thresholds, ranges or morphologies. If the criteria is satisfied for the NF signal and the FF signal, the NF and FF signals are both validated. If either of the NF signal or the FF signal fail to satisfy its respective criteria, e.g., the NF signal 434 is below the NF threshold (T_{LOCAL-NF}) or the FF signal is below the FF threshold (T_{LOCAL-FF}), the NF and FF signals are not validated, i.e., they are declared invalid. When the NF and FF channels both collect NF and FF signals indicative of a validated event of interest from the chamber, then flow moves to 416. At 416, the controller declares a valid local (e.g., ventricular) event at step 416.

Returning to 414, when a validated event is detected over the NF channel, but an invalid event is detected over the FF channel, optionally, flow may branch along dashed line 417. The controller may perform an additional analysis of one or both of the NF and FF signals. The optional additional analysis is indicated in FIG. 4A at 420, at which the controller performs one or more of a morphology analysis, a comparison of the sensed signals to templates, a comparison of the sensed NF and/or FF signals to corresponding thresholds, a comparison of the sensed NF and/or FF signals to corresponding acceptable ranges and the like.

At 422, it is determined whether the analysis at 420 indicates that the NF and FF channels did not detect a validated event of interest. From 422, flow moves to 424 where an invalid or false local event is declared for the local chamber. When the LIMD 100 is in RV, the controller declares a false R-wave at 424. At 428, one or more stimulus pulses may be delivered in accordance with a desired therapy. For example, at 424 when it is determined that no valid ventricular event of interest has occurred, then the LIMD 100 at 428 may pace in the local ventricle, and/or in an adjacent ventricle (e.g., only in the RV, only in the LV or biventricular pacing in the RV and LV). Next flow moves to 418, where the refractory period (RP) or (PVARP) timer is started and flow moves to FIG. 5A.

Alternatively, when the analysis at 420 indicates that the NF and FF channels did detect a valid atrial event, flow moves from 422 along path 426 to 416. At 416, the controller declares a valid local event of interest in the local chamber. Next, at 418, a refractory period timer starts. As one example, when the LIMD 100 is located in the RV, the RP timer represents the post-ventricular atrial refractory period (or PVARP). The RP timer may represent a present or pre-programmed time period, a time period that is automatically updated by the controller and the like. The duration of the PVARP timer is established to represent a post-ventricular atrial refractory period that is desired before the controller will again open the sensing windows to sense for atrial activity. Next, flow moves to FIG. 5A.

FIG. 5A illustrates an exemplary computer implemented processing sequence carried out in accordance with an embodiment to sense for activity originating in an adjacent chamber such as when sensing atrial activity by an LIMD 100 located in the RV or sensing LA activity when the LIMD 100 is located in the LV. Beginning at 510, the process waits for the RP or PVARP timer window to time out or expire, indicating that the RP period of time following the last local event has ended. Next, it is desirable to begin a new sensing session to sense for the next physiologic or normal intrinsic far field event such as an intrinsic atrial event or P-wave occurring in an adjacent chamber.

At 512, the controller activates the NF and FF sensing circuitry to open one or more NF and FF sensing windows to listen for NF and FF signals that are detected over both of the NF and FF channels.

At 514, the FF sensing circuitry in the LIMD 100 listens over the FF channel, such as an atrial channel, for the duration of the FF sensing window. At 516, the NF sensing circuitry listens over the NF channel, such as a ventricular channel, for the duration of the NF sensing window. The NF and FF sensing windows may have the same duration, or differ in length such that one or the other of the NF and FF channels collect sensed signals for a period of time longer than the other of the NF and FF channels. The sensing operations as 514 and 516 are performed simultaneously, or during at least partially overlapping and partially non-overlapping sensing windows, such that the signals sensed over the FF channel and over the NF channel correspond to (and originate from) a same or common cardiac event.

The FF signals are generated or sensed over the FF channel by a first electrode combination that is provided on the LIMD 100 in the local chamber, such as the distal electrode 106 and the intermediate electrode 105. The NF signals are generated or sensed over the NF channel by a second electrode combination that is provided on the LIMD 100 in the local chamber, such as the proximal electrode 104 and the intermediate electrode 105. The electrode combinations may include electrode pairs, single electrodes or sets of more than two electrodes. The electrode combinations used at 514 and 516 may be the same or differ from the electrode combinations discussed above in connection with FIG. 4A.

At 520, and with additional reference to FIG. 5B, the controller compares the signals generated or sensed over the FF channel to a criteria, e.g., a range, threshold and/or morphology template or model to determine whether the FF signals are indicative of a valid intrinsic FF event (e.g., physiologic, normal atrial event). At 522, the controller compares the signals sensed over the NF channel to a range, threshold and/or morphology template or model to determine whether the NF signals are indicative of a valid intrinsic FF event (e.g., physiologic, normal atrial event). By way of example, at 520, the criteria associated with the FF channel may represent a range having an upper threshold (T_{ADJ-FF-UPPER}) (e.g. 0.5 millivolts) and a lower threshold (T_{ADJ-FF-LOWER}) (e.g., 0.2 millivolts). A FF signal 542 above the upper threshold represents an invalid or false signal. A FF signal 544 below the lower threshold also represents an invalid or false signal. A FF signal 540 within the range, i.e., between the upper and lower thresholds, would represent a valid event. In the foregoing example, when the FF signal 542 exceeds the upper threshold, the FF signal is considered to be too strong to have originated from a valid event of interest in the adjacent chamber. Hence, if the threshold is 0.5 millivolts and a FF signal sensed in the FF channel is 0.8 millivolts, the controller may determine that the sensed FF signal could not represent a valid P-wave.

Similarly, the controller compares signals sensed over the NF channel to a NF channel criteria, e.g., amplitude threshold, amplitude range or morphology template. For example, the NF channel threshold associated with a NF channel may be 0.3 millivolts. The controller would determine that a NF signal 546 greater than 0.3 millivolts would represent an invalid P-wave because, by the time an intrinsic event of interest, that occurred in the RA, has propagated to the RV, the signal associated with such an RA intrinsic event would exhibit a very small voltage potential in the apex of the RV (e.g., less than a 0.3 millivolt signal over the NF channel). When the NF channel detects a NF signal 546 greater than the NF channel threshold, it is very unlikely that a corresponding valid intrinsic event of interest originated in the adjacent chamber (e.g., an intrinsic and valid P-wave). When the NF signal 548 is below the threshold, the event is a valid intrinsic event that originates in the adjacent chamber.

Next at 524, the controller determines whether the NF and FF signals sensed over the FF and NF channels satisfy the corresponding thresholds, ranges and/or morphology models. When the NF and FF signals analyzed at 520 and 522 are validated at 524, flow moves to 526 where the controller declares a valid far field event (e.g., P-wave). At 528, the controller then starts a PR timer, such as a timer associated with the AV delay and flow returns to FIG. 4A. The controller then waits the corresponding PR time (AV delay) in FIG. 4A before opening a ventricular sensing window to begin sensing for the next local or ventricular event.

Returning to 524, when the NF and/or FF signals do not satisfy the range, thresholds, morphology or the like, flow moves to 530. At 530 the FF event of interest is declared false. At 532, a therapy is delivered, such as a pacing pulse, from one or more electrodes. Next flow moves to 528 where the PR timer is started. Thereafter the process of FIGS. 4 and 5 is repeated.

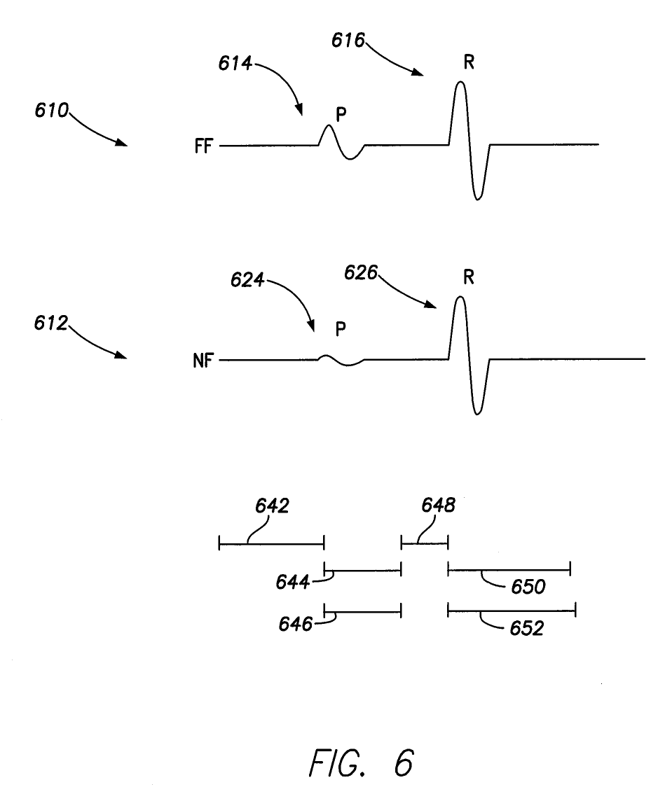

FIG. 6 illustrates an exemplary timing diagram for various sensed signals, sensing windows, and blanking windows, to further explain embodiments herein. In FIG. 6, the signal 610 represents the FF signal sensed over the FF channel, while the signal 612 represents a NF signal sensed over the NF channel. Within the FF signal 610, reference numerals 614 and 616 illustrate signal segments associated with an intrinsic atrial (remote) event and an intrinsic ventricular (local) event, respectively. Within the NF signal 612, reference numerals 624 and 626 illustrate signal segments associated with an intrinsic atrial (remote) event and an intrinsic ventricular (local) event, respectively.

Segment 642 corresponds to a PVARP blanking interval, during which the heart is expected to be in a post ventricular atrial refractory state. During the PVARP interval associated with segment 642, the sensing circuits are deactivated and insensitive to signals detected over the FF and NF channels. The segment 642 corresponds to the RP timer set at 418 (FIG. 4A). At the end of the PVARP blanking interval of segment 642, the sensing circuits are activated for both of the FF and NF channels, as denoted by the FF sense window 644 and NF sense window 646. At the conclusion of the FF and NF sense windows 644 and 646, an AV blanking interval is initiated as denoted by segment 648. During the AV blanking interval of segment 648, the sensing circuits are deactivated which corresponds to the time period of propagation of activity from the atrium to the ventricle (e.g. the AV delay). The segment 648 corresponds to the PR timer set at 528 in FIG. 5A.

Following the AV blanking interval of segment 648, the FF and NF channels are reactivated to initiate FF sensing window 650 and NF sensing window 652. During the FF and NF sensing windows 650 and 652, the sensing circuits collect signals sensed during a local or ventricular event.

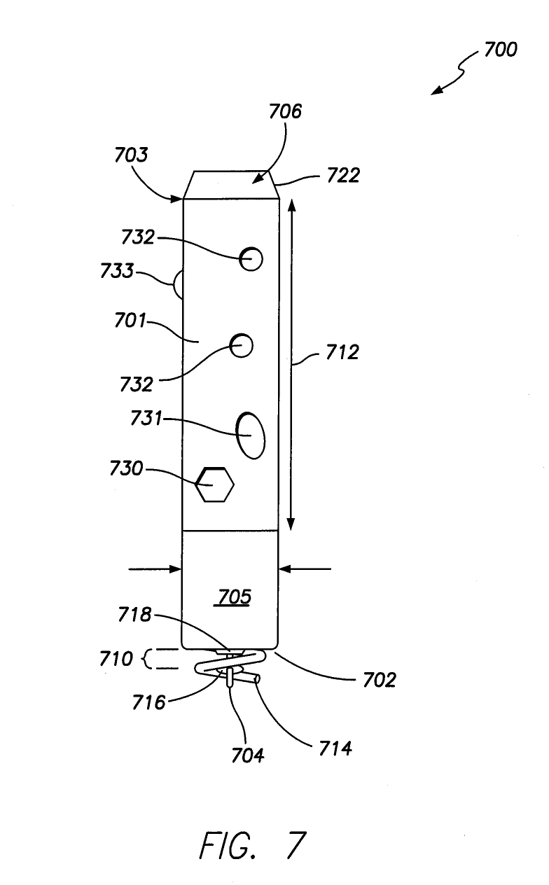

FIG. 7 illustrates an LIMD 700 formed in accordance with an alternative embodiment. The LIMD 700 includes various electrodes such as proximal, intermediate and distal electrodes 704, 705 and 706. The LIMD 700 includes a housing 701 having a base 702 and a top end 703. A proximal electrode 706 is provided on the base 702. The proximal electrode 706 is located on an outer end of a stand-off 718 that extends outward from the base 702. A helical fixation member 714 is provided on the base 702 and configured to be secured to tissue of interest in a local chamber of the heart. The stand-off 718 and proximal electrode 704 are located concentrically within the helical fixation member 714. The proximal electrode 704 is formed as a pin, but may be configured in various other shapes and sizes. An insulation barrier 716 electrically isolates the proximal electrode 704 from the stand-off 718 and the base 702. The trailing edge of the proximal electrode 704 is located an IE spacing 710 from the leading edge of the intermediate electrode 705.

The intermediate electrode 705 is located around the housing 701 and is positioned near the base 702. The intermediate electrode 705 includes leading and trailing edges. The trailing edge is spaced an IE spacing 712 from the leading edge of the distal electrode 706. The distal electrode 706 may have a tapered bezel shape 722.

Optionally, one or more of the proximal, intermediate and distal electrodes 704, 705 and 706 may be omitted and/or supplemented. For example, additional bump electrodes may be positioned about the perimeter of the housing 701 as denoted at 730-733. The bump electrodes 730-733 may have different sizes and shapes.

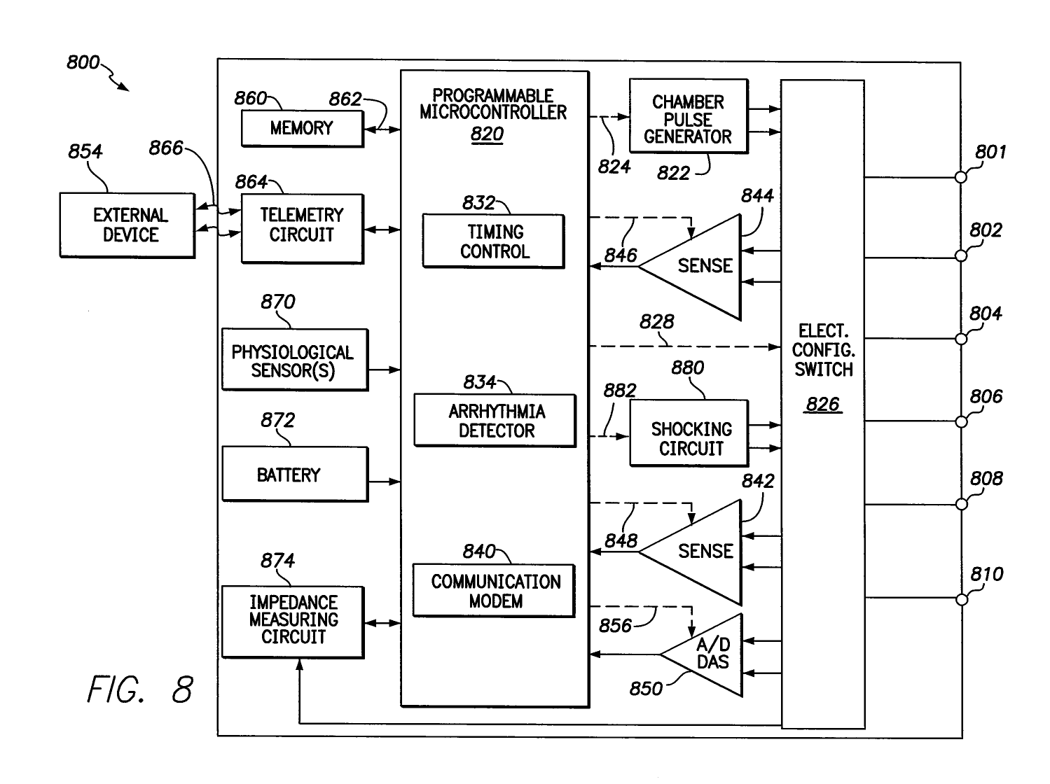

FIG. 8 shows an exemplary LIMD 800 configured for dual-chamber sensing functionality from a primary location within a single chamber of the heart. For example, the LIMD 800 may be implemented as a pacemaker, equipped with both atrial and ventricular sensing circuitry. The LIMD 800 may perform dual chamber pacing. Alternatively, the LIMD 800 may be implemented without atrial pacing. The LIMD 800 may also be implemented to include cardioversion and/or shocking therapy capability.

The LIMD 800 has a housing 801 to hold the electronic/computing components. Housing 801 includes a plurality of terminals 802, 804, 806, 808, 810 that interface with electrodes of the LIMD 800. For example, terminals 802, 804 and 806 may connect to electrodes 104-105, while terminals 808 and 810 are unused. Optionally, the additional terminals 808, 810 may connect with one or more additional electrodes, if available. The type and location of each electrode may vary. For example, the electrodes may include various combinations of ring, tip, coil and shocking electrodes and the like.

The LIMD 800 includes a programmable microcontroller 820 that controls various operations of the LIMD 800, including cardiac monitoring and stimulation therapy. Microcontroller 820 includes a microprocessor (or equivalent control circuitry), RAM and/or ROM memory, logic and timing circuitry, state machine circuitry, and I/O circuitry.

LIMD 800 further includes a first chamber pulse generator 822 that generates stimulation pulses for delivery by one or more electrodes coupled thereto. The pulse generator 822 is controlled by the microcontroller 820 via control signal 824. The pulse generator 822 is coupled to the select electrode(s) via an electrode configuration switch 826, which includes multiple switches for connecting the desired electrodes to the appropriate I/O circuits, thereby facilitating electrode programmability. The switch 826 is controlled by a control signal 828 from the microcontroller 820.

In the example of FIG. 8, a single pulse generator 822 is illustrated. Optionally, the LIMD 800 may include multiple pulse generators, similar to pulse generator 822, where each pulse generator is coupled to one or more electrodes and controlled by the microcontroller 820 to deliver select stimulus pulse(s) to the corresponding one or more electrodes.

Microcontroller 820 is illustrated as including timing control circuitry 832 to control the timing of the stimulation pulses (e.g., pacing rate, PVARP delay, atrio-ventricular (AV) delay etc.). The timing control circuitry 832 may also be used for the timing of refractory periods, blanking intervals, PR timers, RP timers, NF sensing windows, FF sensing windows, noise detection windows, evoked response windows, alert intervals, marker channel timing, and so on. Microcontroller 820 also has an arrhythmia detector 834 for detecting arrhythmia conditions. Although not shown, the microcontroller 820 may further include other dedicated circuitry and/or firmware/software components that assist in monitoring various conditions of the patient's heart and managing pacing therapies.

The LIMD 800 includes sensing circuitry that includes one or more sensing circuits such as sensing circuits 842 and 844 selectively coupled to one or more electrodes through the switch 826. The functionality of the sensing circuits 842 and 844 may be performed by one, two or more circuits. The sensing circuits 842 and 844 detect the presence of cardiac activity in local and remote chambers of the heart. Each of the sensing circuits 842 and 844 may include dedicated sense amplifiers, multiplexed amplifiers, or shared amplifiers. The sensing circuits 842 and 844 may further employ one or more low power, precision amplifiers with programmable gain and/or automatic gain control, bandpass filtering, and threshold detection circuit to selectively sense the cardiac signal of interest. The automatic gain control enables sensing of low amplitude signal characteristics, such as with atrial fibrillation, far field signals and the like.

The switch 826 connects the sensing circuits 842 and 844 to select combinations of the electrodes. For example, the sensing circuit 842 may be connected to electrodes 104 and 105, while sensing circuit 844 is connected to electrodes 105 and 106. The sensing circuits 842 and 844 output NF and FF sense signals based on various sensing modes and sensing parameters, such as bipolar, monopolar and the like. The NF and FF signals may represent a difference between the voltage potentials detected by the corresponding electrodes 104-106.

The sensing circuits 842 and 844 define the NF and FF channels, respectively. The sensing circuits 842 and 844 are activated and deactivate by the control signals 848 and 846, from the controller 820. The sensing circuits 842 and 844 are activated during sensing windows by the controller 820 based on timing parameters that may be programmed by a physician and/or by a device manufacturer. The timing parameters may be periodically or automatically updated. The timing parameters may be automatically updated by the LIMD 100 based on baseline or real time cardiac signals, physiologic measurements, patient behavior and the like.

As one example, an atrial event (FF) sensing window may be initiated or activated after a predetermined interval following an R-wave (e.g., the post ventricular atrial refractory period or PVARP interval). Also, a ventricular event (NF) sensing window may be initiated or activated a predetermined interval following a P-wave (e.g., the AV delay). The lengths of the atrial and ventricular sensing windows will differ and may be programmable and/or automatically updated by the LIMD 100.

Switch 826 determines the sensing polarity of the cardiac signal sensed by each of the sensing circuits 842 and 844 by selectively closing the appropriate switches. In this way, the clinician may program the sensing polarity independent of the stimulation polarity. The clinician may also program the sensing polarity of the sensing circuit 842 to be different from the sensing polarity of the sensing circuit 844. Optionally, alternative combinations of electrodes may connect to the sensing circuits 842 and 844.

During the atrial and ventricular event sensing windows, the sensing circuits 842 and 844 output corresponding sensed signals. The sensed signals are analyzed by the controller 820 to determine whether valid P-wave and/or R-wave events of interest have occurred. The output of the sensing circuits 842 and 844 are connected to the microcontroller 820 which, in turn, triggers or inhibits one or more pulse generators 822 in response to the absence or presence of cardiac activity. The sensing circuits 842 and 844 receive control signals 848 and 846 from the microcontroller 820 for the purposes of controlling activation and deactivation of sensing windows. The control signals 848 and 846 from the microcontroller 820 may also be used to control the gain, threshold, polarization charge removal circuitry (not shown), and the timing of any blocking circuitry (not shown) coupled to the inputs of the sensing circuit 842 and 844.

In the example of FIG. 8, a pair of single sensing circuits 842 and 844 is illustrated. Optionally, the LIMD 800 may include multiple sensing circuit, similar to sensing circuits 842 and 844, where each sensing circuit is coupled to one or more electrodes and controlled by the microcontroller 820 to sense electrical activity detected at the corresponding one or more electrodes. The sensing circuit 844 may operate in a unipolar sensing configuration or in a bipolar sensing configuration.

The LIMD 800 further includes an analog-to-digital (A/D) data acquisition system (DAS) 850 coupled to one or more electrodes via the switch 826 to sample cardiac signals across any pair of desired electrodes. The data acquisition system 850 is configured to acquire intracardiac electrogram signals, convert the raw analog data into digital data, and store the digital data for later processing and/or telemetric transmission to an external device 854 (e.g., a programmer, local transceiver, or a diagnostic system analyzer). The data acquisition system 850 is controlled by a control signal 856 from the microcontroller 820.

The microcontroller 820 is coupled to a memory 860 by a suitable data/address bus 862. The programmable operating parameters used by the microcontroller 820 are stored in memory 860 and used to customize the operation of the LIMD 800 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart 808 within each respective tier of therapy.

The operating parameters of the LIMD 800 may be non-invasively programmed into the memory 860 through a telemetry circuit 864 in telemetric communication via communication link 866 with the external device 854. The telemetry circuit 864 allows intracardiac electrograms and status information relating to the operation of the LIMD 800 (as contained in the microcontroller 820 or memory 860) to be sent to the external device 854 through the established communication link 866.

The IMD 802 can further include magnet detection circuitry (not shown), coupled to the microcontroller 820, to detect when a magnet is placed over the unit. A magnet may be used by a clinician to perform various test functions of the unit 802 and/or to signal the microcontroller 820 that the external programmer 854 is in place to receive or transmit data to the microcontroller 820 through the telemetry circuits 864.

The LIMD 800 may be equipped with a communication modem (modulator/demodulator) 840 to enable wireless communication with a remote device, such as a second implanted LIMD in a master/slave arrangement, such as described in U.S. Pat. No. 7,630,767. In one implementation, the communication modem 840 uses high frequency modulation. As one example, the modem 840 transmits signals between a pair of LIMD electrodes, such as between the housing 801 and any one of the electrodes connected to terminals 802-810. The signals are transmitted in a high frequency range of approximately 20-80 kHz, as such signals travel through the body tissue in fluids without stimulating the heart or being felt by the patient. The communication modem 840 may be implemented in hardware as part of the microcontroller 820, or as software/firmware instructions programmed into and executed by the microcontroller 820. Alternatively, the modem 840 may reside separately from the microcontroller as a standalone component.

The LIMD 800 can further include one or more physiologic sensors 870. Such sensors are commonly referred to as “rate-responsive” sensors because they are typically used to adjust pacing stimulation rates according to the exercise state of the patient. However, the physiological sensor 870 may further be used to detect changes in cardiac output, changes in the physiological condition of the heart, or diurnal changes in activity (e.g., detecting sleep and wake states). Signals generated by the physiological sensors 870 are passed to the microcontroller 820 for analysis. The microcontroller 820 responds by adjusting the various pacing parameters (such as rate, AV Delay, V-V Delay, etc.) at which the atrial and ventricular pacing pulses are administered. While shown as being included within the housing 801, the physiologic sensor(s) 870 may be external to the housing 801, yet still be implanted within or carried by the patient. Examples of physiologic sensors might include sensors that, for example, sense respiration rate, pH of blood, ventricular gradient, activity, position/posture, temperature, minute ventilation (MV), and so forth.

A battery 872 provides operating power to all of the components in the LIMD 800. The battery 872 is capable of operating at low current drains for long periods of time, and is capable of providing high-current pulses (for capacitor charging) when the patient requires a shock pulse (e.g., in excess of 2 A, at voltages above 2 V, for periods of 10 seconds or more). The battery 872 also desirably has a predictable discharge characteristic so that elective replacement time can be detected.

The LIMD 800 further includes an impedance measuring circuit 874, which can be used for many things, including: impedance surveillance during the acute and chronic phases for proper LIMD positioning or dislodgement; detecting operable electrodes and automatically switching to an operable pair if dislodgement occurs; measuring respiration or minute ventilation; measuring thoracic impedance; detecting when the device has been implanted; measuring stroke volume; and detecting the opening of heart valves; and so forth. The impedance measuring circuit 874 is coupled to the switch 826 so that any desired electrode may be used.

The LIMD 800 may optionally include a shocking circuit 880 controlled by way of a control signal 882. The shocking circuit 880 generates shocking pulses of low (e.g., up to 0.5 joules), moderate (e.g., 0.5-10 joules), or high energy (e.g., 811 to 40 joules), as controlled by the microcontroller 820. Such shocking pulses are applied to the patient's heart 808 through shocking electrodes, if available on the LIMD. It is noted that the shock therapy circuitry is optional and may not be implemented in the LIMD, as the various LIMDs described above and further below will typically not be configured to deliver high voltage shock pulses. On the other hand, it should be recognized that an LIMD may be used within a system that includes backup shock capabilities, and hence such shock therapy circuitry may be included in the LIMD.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A leadless intra-cardiac medical device (LIMD) configured to be implanted entirely within a single local chamber of the heart and remote from an adjacent chamber of the heart, the LIMD comprising:
a housing having a proximal end configured to engage local tissue of interest in the local chamber and a distal end;
electrodes located at multiple locations along the housing;
sensing circuitry configured to define a far field (FF) channel between a first combination of the electrodes to sense FF signals;
the sensing circuitry configured to define a near field (NF) channel between a second combination of the electrodes to sense NF signals; and
a controller configured to analyze the NF and FF signals to determine whether the NF and FF signals collectively indicate that a validated event of interest occurred in the adjacent chamber.

2. The LIMD of claim 1, wherein the electrodes include a proximal electrode located at the proximal end, a distal electrode located at the distal end, and an intermediate electrode located at an intermediate region along the housing.

3. The LIMD of claim 2, wherein the first electrode combination comprises the distal electrode and the intermediate electrode.

4. The LIMD of claim 2, wherein the second electrode combination comprises the proximal electrode and the intermediate electrode.

5. The LIMD of claim 2, wherein the proximal and intermediate electrodes are separated by a first inter-electrode (IE) spacing, the distal and intermediate electrodes are separated by a second IE spacing, and the second IE spacing is greater than the first IE spacing.

6. The LIMD of claim 1, wherein the controller is configured to compare the FF signals sensed over the FF channel to a FF adjacent-chamber criteria, compare the NF signals sensed over the NF channel to a NF adjacent-chamber criteria, and declare a validated event when both of the criteria are satisfied.

7. The LIMD of claim 6, wherein the FF adjacent-chamber criteria is a FF signal amplitude threshold, the NF adjacent-chamber criteria is a NF signal amplitude threshold and the criteria is satisfied when the amplitude of the sensed FF signals exceeds the FF signal amplitude threshold and the amplitude of the sensed NF signals does not exceed the NF signal amplitude threshold.

8. The LIMD of claim 6, wherein the FF adjacent-chamber criteria is a FF signal morphology, the NF adjacent-chamber criteria is a NF signal morphology, and the criteria is satisfied when the morphology of the sensed FF signals matches the FF signal morphology and the morphology of the sensed NF signals matches the NF signal morphology.

9. The LIMD of claim 6, wherein the FF adjacent-chamber criteria is a FF signal amplitude range, the NF adjacent-chamber criteria is a NF signal amplitude threshold, and the criteria is satisfied when the amplitude of the sensed FF signals is within the FF signal amplitude range and the amplitude of the sensed NF signals does not exceed the NF signal amplitude threshold.

10. The LIMD of claim 1, wherein the controller is further configured to analyze the NF and FF signals to determine whether the NF and FF signals collectively indicate that a validated event of interest occurred in the local chamber.

11. The LIMD of claim 10, wherein the controller is configured to compare the FF signals sensed over the FF channel to a FF local-chamber criteria, compare the NF signals sensed over the NF channel to a NF local-chamber criteria, and declare a validated event when both of the criteria are satisfied.

12. The LIMD of claim 11, wherein the FF local-chamber criteria is a FF signal amplitude threshold, the NF local-chamber criteria is a NF signal amplitude threshold, and the criteria is satisfied when the amplitude of the sensed FF signals exceeds the FF signal amplitude threshold and the amplitude of the sensed NF signals exceeds the NF signal amplitude threshold.

13. The LIMD of claim 11, wherein the FF local-chamber criteria is a FF signal morphology, the NF local chamber criteria is a NF signal morphology, and the criteria is satisfied when the morphology of the sensed FF signals matches the FF signal morphology and the morphology of the sensed NF signals match the NF signal morphology.

14. A method for sensing cardiac activity from a leadless intra-cardiac medical device (LIMD) configured to be implanted entirely within a single local chamber of the heart and remote from an adjacent chamber, the method comprising:
sensing far field (FF) signals over a FF channel between a first combination of electrodes provided on the LIMD in the local chamber;
sensing near field (NF) signals over a NF channel between a second combination of electrodes provided on the LIMD in the local chamber; and
analyzing both of the NF and FF signals to determine whether the NF and FF signals indicate that a validated event of interest occurred in the adjacent chamber.

15. The method of claim 11, wherein analyzing comprises:
comparing the FF signals sensed over the FF channel to a FF adjacent-chamber criteria;
comparing the NF signals sensed over the NF channel to a NF adjacent-chamber criteria; and
declaring a validated event when both of the criteria are satisfied.

16. The method of claim 15, wherein the FF adjacent-chamber criteria is a FF signal amplitude threshold, the NF adjacent-chamber criteria is a NF signal amplitude threshold, and the criteria is satisfied when the amplitude of the sensed FF signals exceeds the FF signal amplitude threshold and the amplitude of the sensed NF signals does not exceed the NF signal amplitude threshold.

17. The method of claim 15, wherein the FF adjacent-chamber criteria is a FF signal morphology, the NF adjacent-chamber criteria is a NF signal morphology, and the criteria is satisfied when the morphology of the sensed FF signals matches the FF signal morphology and the morphology of the sensed NF signals matches the NF signal morphology.

18. The method of claim 15, wherein the FF adjacent-chamber criteria is a FF signal amplitude range, the NF adjacent-chamber criteria is a NF signal amplitude threshold, and the criteria is satisfied when the amplitude of the sensed FF signals is within the FF signal amplitude range and the amplitude of the sensed NF signals does not exceed the NF signal amplitude threshold.

19. The method of claim 11, further comprising analyzing the NF and FF signals to determine whether the NF and FF signals collectively indicate that a validated event of interest occurred in the local chamber.

20. The method of claim 19, wherein analyzing comprises:
comparing the FF signals sensed over the FF channel to a FF local-chamber criteria;
comparing the NF signals sensed over the NF channel to a NF local-chamber criteria; and
declaring a validated event when both of the criteria are satisfied.

21. The method of claim 20, wherein the FF local-chamber criteria is a FF signal amplitude threshold, the NF local-chamber criteria is a NF signal amplitude threshold, and the criteria is satisfied when the amplitude of the sensed FF signals exceeds the FF signal amplitude threshold and the amplitude of the sensed NF signals exceeds the NF signal amplitude threshold.

22. The method of claim 20, wherein the FF local-chamber criteria is a FF signal morphology, the NF local chamber criteria is a NF signal morphology, and the criteria is satisfied when the morphology of the sensed FF signals matches the FF signal morphology and the morphology of the sensed NF signals matches the NF signal morphology.
